(19) United States
(12) Patent Application Publication
(10) Pub. No.: US 2008/0275346 A1
(43) Pub. Date: Nov. 6, 2008

(54) ULTRASOUND THERAPY APPARATUS

(76) Inventors: Po-Hsing LEE, Taipei Hsien (TW); Chia-Chen HO, Ping-Tung Hsien (TW)

Correspondence Address: OSTROLENK FABER GERB & SOFFEN, 1180 AVENUE OF THE AMERICAS, NEW YORK, NY, 100368403, US

(21) Appl. No.: 11/744,445

(22) Filed: May 4, 2007

(51) Int. Cl. A61B 8/00

(52) U.S. Cl. 600/459

(57) ABSTRACT

An ultrasound therapy apparatus includes a signal generating device for generating an oscillatory wave signal, and configured to switch automatically frequency of the oscillatory wave signal between at least first and second preset frequency values. An amplitude adjusting unit is connected electrically to the signal generating device for receiving the oscillatory wave signal therefrom, and is operable so as to adjust amplitude of the oscillatory wave signal for outputting a driving signal. A probe device is connected electrically to the amplitude adjusting unit, and is driven by the driving signal from the amplitude adjusting unit so as to output ultrasound energy that is adapted to be applied to a human body.

### BACKGROUND OF THE INVENTION

1. Field of the Invention

The invention relates to an ultrasound apparatus, more particularly to an ultrasound therapy apparatus.

2. Description of the Related Art

Referring to FIG. 1, a conventional ultrasound therapy apparatus is shown to include an operating interface 91, a control module 92, an amplitude adjusting module 93, and a probe device 94.

The operating interface 91 is operable so as to output a control signal representative of duration of therapy. The control module 92 is connected electrically to the operating interface 91 for receiving the control signal therefrom, and is configured to generate an oscillatory wave signal (v1′) within the duration of therapy. The amplitude adjusting module 93 is connected electrically to the control module 92 for receiving the oscillatory wave signal (v1′) therefrom, and adjusts amplitude of the oscillatory wave signal (v1′) for outputting a driving signal (v2′) that has amplitude equal to about 30 volts. The probe device 94 includes an ultrasound transducer 941, such as a piezoelectric oscillator, connected electrically to the amplitude adjusting module 93 and driven by the driving signal (v2′) so as to output ultrasound energy that has a frequency corresponding to that of the driving signal (v2′) and that is applied to a human body for physical therapy.

When the frequency of the ultrasound energy is about 3 MHz, the ultrasound energy has superior therapeutic effect for the human body within a range from the skin surface of the human body to about 1 cm below the skin surface of the human body. When the frequency of ultrasound energy is about 1 MHz, the ultrasound energy has superior therapeutic effect for the human body within a range from 1 cm below the skin surface of the human body to 3 cm below the skin surface of the human body. As such, the ultrasound energy is required to have frequencies of 1 MHz and 3 MHz. However, in the conventional ultrasound therapy apparatus, the oscillatory wave signal (v1′) is switched between first and second preset frequency values, such as 1 MHz and 3 MHz, under manual control, thereby resulting in inconvenience during use.

Furthermore, rising temperature of a probe electrode unit (not shown) of the probe device after a long period of use may result in injury to the human body.

### SUMMARY OF THE INVENTION

Therefore, the object of the present invention is to provide an ultrasound therapy apparatus that can output ultrasound energy with a frequency automatically switched between at least first and second frequency values.

According to the present invention, an ultrasound therapy apparatus comprises:

a signal generating device for generating an oscillatory wave signal, and configured to switch automatically frequency of the oscillatory wave signal between at least first and second preset frequency values;

an amplitude adjusting unit connected electrically to the signal generating device for receiving the oscillatory wave signal therefrom, and operable so as to adjust amplitude of the oscillatory wave signal for outputting a driving signal; and

a probe device connected electrically to the amplitude adjusting unit and driven by the driving signal from the amplitude adjusting unit so as to output ultrasound energy that is adapted to be applied to a human body.

### BRIEF DESCRIPTION OF THE DRAWINGS

Other features and advantages of the present invention will become apparent in the following detailed description of the preferred embodiment with reference to the accompanying drawings, of which:

FIG. 1 is a schematic circuit block diagram illustrating a conventional ultrasound therapy apparatus;

FIG. 2 is a schematic circuit block diagram illustrating the preferred embodiment of an ultrasound therapy apparatus according to the present invention;

FIGS. 3 and 4 are plots of waveforms of a driving signal generated by an amplitude adjusting unit of the preferred embodiment; and

FIGS. 5 and 6 are flow charts illustrating how the preferred embodiment is operated to output the ultrasound energy.

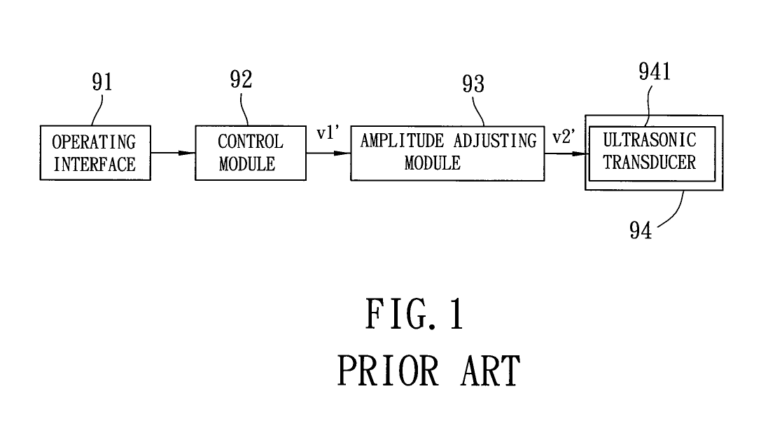

### DETAILED DESCRIPTION OF THE PREFERRED EMBODIMENT

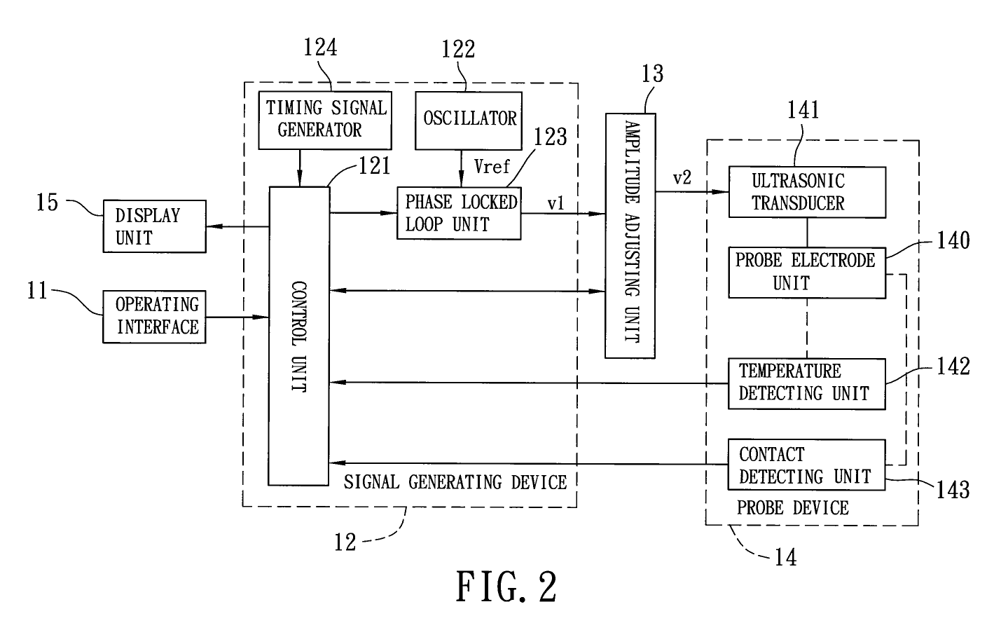

Referring to FIG. 2, the preferred embodiment of an ultrasound therapy apparatus according to the present invention is shown to include a signal generating device 12, an amplitude adjusting unit 13, a probe device 14, an operating interface 11, and a display unit 15.

The signal generating device 12 generates an oscillatory wave signal (v1), which has a fixed amplitude, and is configured to switch automatically frequency of the oscillatory wave signal (v1) between first and second preset frequency values. In this embodiment, the first and second preset frequency values are respectively 1 MHz and 3 MHz. The signal generating device 12 includes an oscillator 122, a control unit 121, a phase locked loop unit 123 and a timing signal generator 124.

The oscillator 122 generates a reference oscillatory wave signal (Vref) having a reference oscillation frequency.

The timing signal generator 124 generates a timing signal having a frequency of 10 MHz.

The control unit 121 is connected electrically to the timing signal generator 124, and the timing signal from the timing signal generator 124 serves as a clock signal of the control unit 121. The control unit 121 generates a frequency divisor that is automatically switched between two preset divisor values.

The phase locked loop unit 123 is connected electrically to the oscillator 122 and the control unit 121, and is operable so as to output the oscillatory wave signal (v1) in response to the reference oscillatory wave signal (Vref) from the oscillator 122 and the frequency divisor from the control unit 121, wherein the oscillatory wave signal (v1) is obtained by dividing the reference oscillation frequency of the reference oscillatory wave signal (Vref) by the frequency divisor from the control unit 121.

The amplitude adjusting unit 13 is connected electrically to the phase locked loop unit 123 and the control unit 121 of the signal generating device 12 for receiving the oscillatory wave signal (v1) from the phase locked loop unit 123, and is operable so as to adjust amplitude of the oscillatory wave signal (v1) for outputting a driving signal (v2). In this embodiment, the control unit 121 of the signal generating device 12 further detects output power outputted by the amplitude adjusting unit 13, and controls the amplitude adjusting unit 13 to output target output power based on the output power detected by the control unit 121 in a known manner.

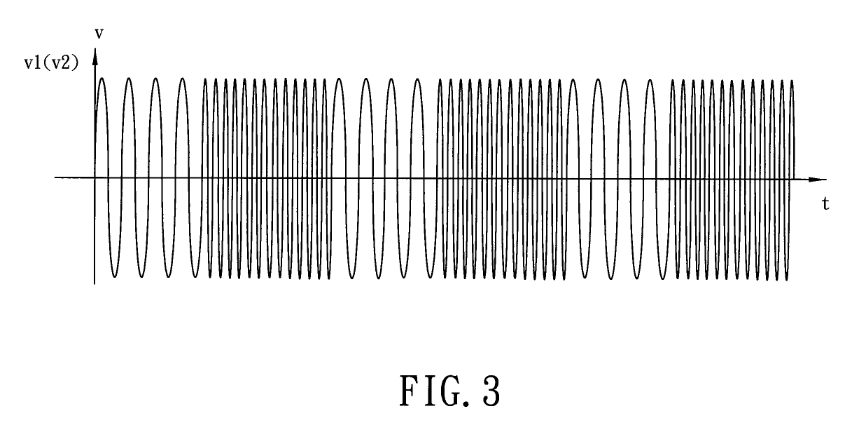

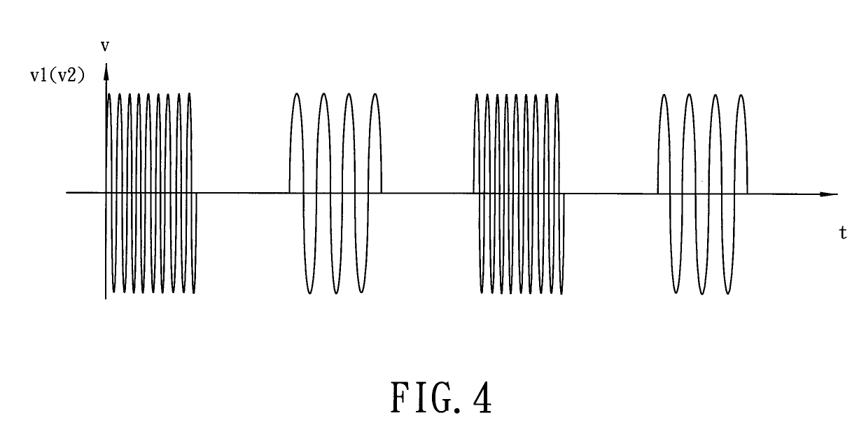

The operating interface 11 is connected electrically to the control unit 121 of the signal generating device 12, and is operable so as to output a control signal representative of duration of therapy to the control unit 121. As such, the signal generating device 12 controls duration of output of the oscillatory wave signal (v1) based on the control signal from the operating interface 11. Preferably, the operating interface 11 is further operable to control respective duration of output of the oscillatory wave signal (v1) by the signal generating device 12 at each of the preset frequency values. In one example of the oscillatory wave signal (v1) shown in FIG. 3, the oscillatory wave signal (v1) includes a plurality of first signal portions temporally spaced apart from each other, and a plurality of second signal portions temporally spaced apart from each other. The frequency of the first signal portions is the first preset frequency value, i.e., 1 MHz. The frequency of the second signal portions is the second preset frequency value, i.e., 3 MHz. The first and second signal portions are temporally interleaved with each other. The oscillatory wave signal (v1) is a continuous signal. FIG. 4 shows an alternative example of the oscillatory wave signal (v1) that differs from that of FIG. 3 in that the oscillatory wave signal (v1) is a discontinuous signal. In this embodiment, the operating interface 11 includes a plurality of function keys (not shown), such as a setup key, a stop key, a start key, etc.

The display unit 15 is connected electrically to the control unit 121 of the signal generating device 12 for displaying information related to therapy duration.

The probe device 14 is connected electrically to the amplitude adjusting unit 13, and is driven by the driving signal (v2) from the amplitude adjusting unit 13 so as to output ultrasound energy that is adapted to be applied to a human body. In this embodiment, the probe device 14 includes a probe electrode unit 140, an ultrasound transducer 141, a temperature detecting unit 142, and a contact detecting unit 143.

The probe electrode unit 140 is adapted to be placed in contact with the human body.

The ultrasound transducer 141, such as a piezoelectric oscillator, is connected electrically to the amplitude adjusting unit 13 and the probe electrode unit 140, and is operable so as to convert the driving signal (v2) from the amplitude adjusting unit 13 into the ultrasound energy and so as to output the ultrasound energy via the probe electrode unit 140. It is noted that the frequency of the ultrasound energy corresponds to that of the driving signal (v2).

The temperature detecting unit 142 is connected electrically to the control unit 121 of the signal generating device 12, and is operable so as to detect temperature of the probe electrode unit 140 in a known manner and so as to alert the control unit 121 of the signal generating device 12 when the temperature of the probe electrode unit 140 is higher than a preset threshold. The control unit 121 responds to alert of the temperature detecting unit 142 by controlling the amplitude adjusting unit 13 to terminate output of the driving signal (V2), thereby effectively avoiding injury to the human body due to an excessively high temperature of the probe electrode unit 140.

The contact detecting unit 143 is connected electrically to the control unit 121 of the signal generating device 12, and is operable so as to detect contact between the probe electrode unit 140 and the human body in a known manner and so as to alert the control unit 121 of the signal generating device 12 when the probe electrode unit 140 does not contact the human body. The control unit 121 responds to alert of the contact detecting unit 143 by controlling the amplitude adjusting unit 13 to terminate output of the driving signal (v2), thereby conserving power consumption of the ultrasound therapy apparatus.

In such a configuration, the control unit 121 generates the frequency divisor that is automatically switched between two preset divisor values in response to the control signal from the operating interface 11 such that the frequency of the oscillatory wave signal (v1) generated by the signal generating device 12 can be automatically switched between 1 MHz and 3 MHz. Thus, the ultrasound energy having the frequency automatically switched between 1 MHz and 3 MHz can be obtained, thereby resulting in convenience during use.

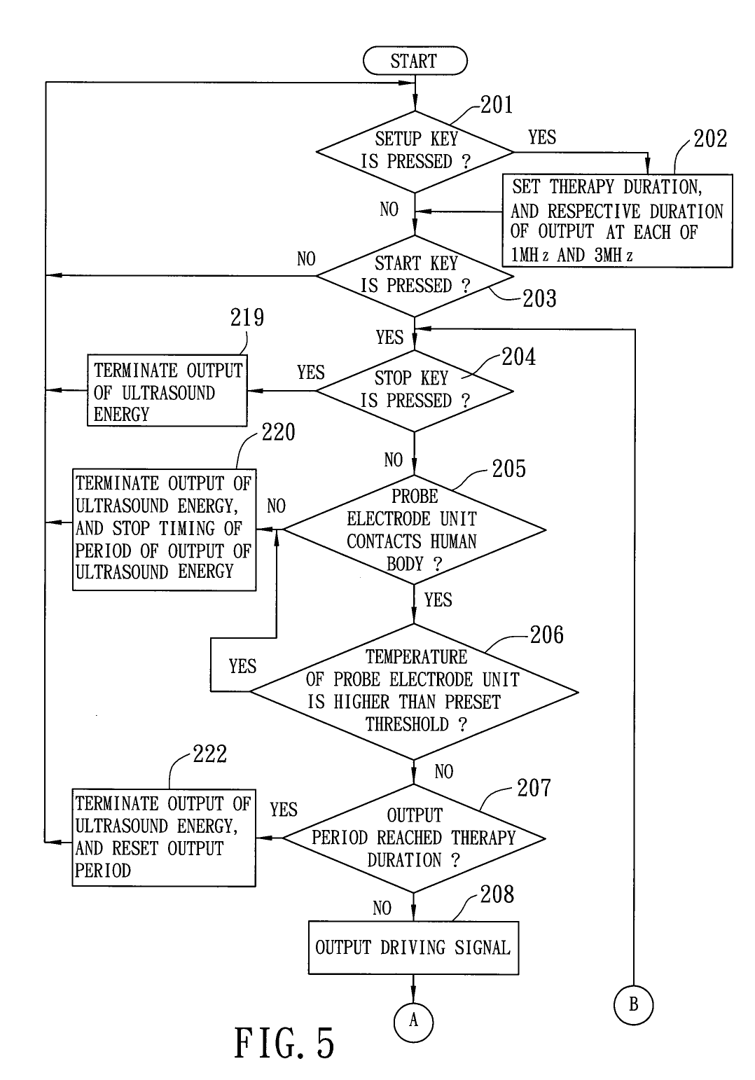

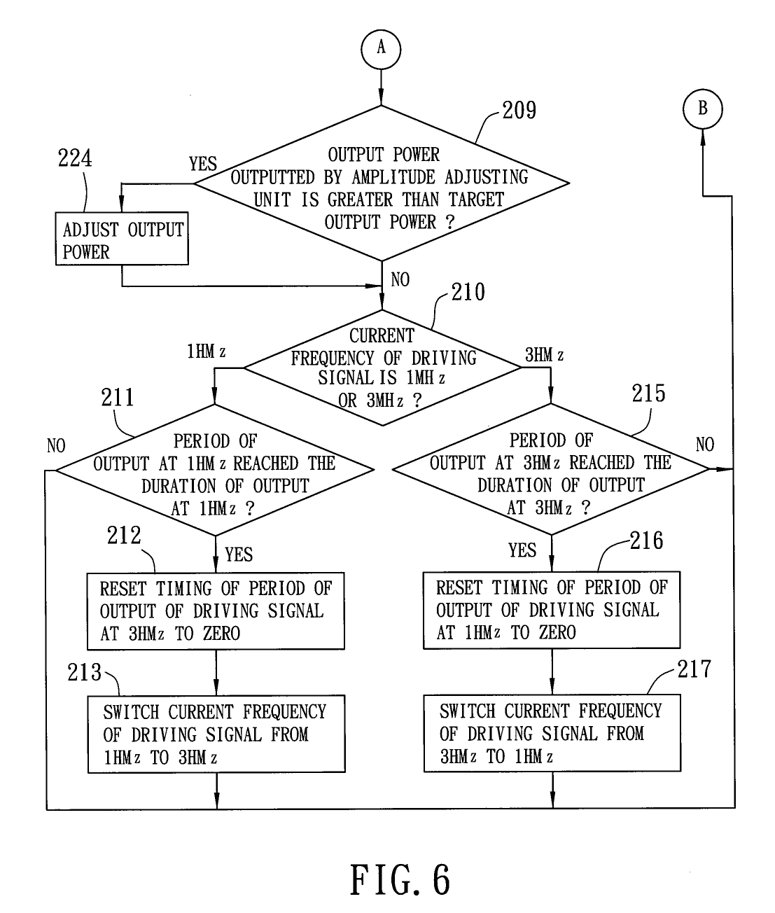

Referring to FIGS. 5 and 6, there are shown flow charts to illustrate how the ultrasound therapy apparatus is operated to output the ultrasound energy that has the frequency automatically switched between 1 MHz and 3 MHz. In step 201, the control unit 121 is configured to determine whether the setup key is pressed. If negative, the flow proceeds to step 203. Otherwise, the flow goes to step 202. In step 202, the control unit 121 is configured through the operating interface 11 to set therapy duration, and respective duration of output of the ultrasound energy at each of 1 MHz and 3 MHz based on the control signal outputted by the operating interface 11, and the flow proceeds to step 203. In step 203, the control unit 121 is configured to determine whether the start key is pressed. If negative, the flow goes back to step 201. Otherwise, the flow goes to step 204. In step 204, the control unit 121 is configured to determine whether the stop key is pressed. If affirmative, the flow proceeds to step 219. In step 219, the control unit 121 controls the amplitude adjusting unit 13 to terminate the output of the driving signal (v2), thereby terminating output of the ultrasound energy, and the flow goes back to step 201. When it is determined in step 204 that the stop key is not pressed, the flow goes to step 205, where the contact detecting unit 143 detects whether the probe electrode unit 140 contacts the human body. If negative, the flow proceeds to step 220. In step 220, the control unit 121 controls the amplitude adjusting unit 13 to terminate the output of the driving signal (v2), thereby terminating the output of the ultrasound energy, and stops timing of a period of the output of the ultrasound energy, and the flow goes back to step 201. When it is determined in step 205 that the probe electrode unit 140 contacts the human body, the flow goes to step 206, where the temperature detecting unit 142 detects whether the temperature of the probe electrode unit 140 is higher than the preset threshold. If affirmative, the flow goes to step 220. Otherwise, the flow goes to step 207. In step 207, the control unit 121 determines whether the period of the output of the ultrasound energy has reached the therapy duration set in step 202. If affirmative, the flow proceeds to step 222. In step 222, the control unit 121 controls the amplitude adjusting unit 13 to terminate the output of the driving signal (v2), thereby terminating the output of the ultrasound energy, and resets the output period to zero, and the flow goes back to step 201. When it is determined in step 207 that the output period has not yet reached the therapy duration, the flow goes to step 208, where the amplitude adjusting unit 13 adjusts amplitude of the oscillatory wave signal (v1) for outputting the driving signal (V2). In step 209, the control unit 121 detects whether the output power outputted by the amplitude adjusting unit 13 is greater than the target output power. If negative, the flow proceeds to step 210. Otherwise, the flow goes to step 224. In step 224, the output power is adjusted by the amplitude adjusting unit 13 to meet the target output power, and the flow goes to step 210. In step 210, the control unit 121 judges whether the current frequency of the driving signal (v2) is 1 MHz or 3 MHz. In step 211, when the current frequency of the driving signal (v2) is 1 MHz, the control unit 121 determines whether the period of the output of the driving signal (v2) at 1 MHz reached the duration of the output of the ultrasound energy at 1 MHz that was set in step 202. If negative, the flow goes back to step 204. Otherwise, the flow goes to step 212. In step 212, the control unit 121 resets timing of the period of the output of the driving signal (v2) at 3 MHz to zero. In step 213, the signal generating device 12 is configured to switch the current frequency of the driving signal (v2) automatically from 1 MHz to 3 MHz, and the flow goes back to step 204. In step 215, when the current frequency of the driving signal (v2) is 3 MHz, the control unit 121 determines whether the period of the output of the driving signal (v2) at 3 MHz reached the duration of the output of the ultrasound energy at 3 MHz that was set in step 202. If negative, the flow goes back to step 204. Otherwise, the flow goes to step 216. In step 216, the control unit 121 resets timing of the period of the output of the driving signal (v2) at 1 MHz to zero. In step 217, the signal generating device 12 is configured to switch the current frequency of the driving signal (v2) automatically from 3 MHz to 1 MHz, and the flow goes back to step 204.

To sum up, the ultrasound therapy apparatus of this invention can output the ultrasound energy having the frequency automatically switched between two preset frequency values, thereby resulting in convenience during use. Furthermore, due to the presence of the temperature detecting unit 142, injury to the human body due to an excessively high temperature of the probe electrode unit 140 can be avoided.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

We claim:

1. An ultrasound therapy apparatus comprising:
a signal generating device for generating an oscillatory wave signal, and configured to switch automatically frequency of the oscillatory wave signal between at least first and second preset frequency values;
an amplitude adjusting unit connected electrically to said signal generating device for receiving the oscillatory wave signal therefrom, and operable so as to adjust amplitude of the oscillatory wave signal for outputting a driving signal; and
a probe device connected electrically to said amplitude adjusting unit and driven by the driving signal from said amplitude adjusting unit so as to output ultrasound energy that is adapted to be applied to a human body.

2. The ultrasound therapy apparatus as claimed in claim 1, wherein said signal generating device includes:
an oscillator for generating a reference oscillatory wave signal having a reference oscillation frequency;
a control unit for generating a frequency divisor that is automatically switched between at least two preset divisor values; and
a phase locked loop unit connected electrically to said oscillator, said control unit and said amplitude adjusting unit, said phase locked loop unit being operable so as to output the oscillatory wave signal in response to the reference oscillatory wave signal and the frequency divisor, wherein the oscillatory wave signal is obtained by dividing the reference oscillation frequency of the reference oscillatory wave signal by the frequency divisor from said control unit.

3. The ultrasound therapy apparatus as claimed in claim 2, wherein said probe device includes:
a probe electrode unit adapted to be placed in contact with the human body;
an ultrasound transducer connected electrically to said amplitude adjusting unit and said probe electrode unit, and operable so as to convert the driving signal from said amplitude adjusting unit into the ultrasound energy and so as to output the ultrasound energy via said probe electrode unit; and
a temperature detecting unit connected electrically to said control unit of said signal generating device, and operable so as to detect temperature of said probe electrode unit and so as to alert said control unit of said signal generating device when the temperature of said probe electrode unit is higher than a preset threshold, said control unit responding to alert of said temperature detecting unit by controlling said amplitude adjusting unit to terminate output of the driving signal.

4. The ultrasound therapy apparatus as claimed in claim 3, wherein said probe device further includes a contact detecting unit connected electrically to said control unit of said signal generating device, and operable so as to detect contact between said probe electrode unit and the human body and so as to alert said control unit of said signal generating device when said probe electrode unit does not contact the human body, said control unit responding to alert of said contact detecting unit by controlling said amplitude adjusting unit to terminate output of the driving signal.

5. The ultrasound therapy apparatus as claimed in claim 2, wherein said control unit of said signal generating device is further connected electrically to said amplitude adjusting unit for detecting output power outputted by said amplitude adjusting unit and for controlling said amplitude adjusting unit to output target output power based on the output power detected by said control unit.

6. The ultrasound therapy apparatus as claimed in claim 1, further comprising an operating interface connected electrically to said signal generating device and operable so as to output a control signal representative of duration of therapy to said signal generating device, said signal generating device controlling duration of output of the oscillatory wave signal based on the control signal from said operating interface.

7. The ultrasound therapy apparatus as claimed in claim 6, wherein said operating interface is further operable to control respective duration of output of the oscillatory wave signal by said signal generating device at each of the preset frequency values.

8. The ultrasound therapy apparatus as claimed in claim 1, wherein the oscillatory wave signal includes a plurality of first signal portions temporally spaced apart from each other, and a plurality of second signal portions temporally spaced apart from each other, the frequency of the first signal portions being the first preset frequency value, the frequency of the second signal portions being the second preset frequency value.

9. The ultrasound therapy apparatus as claimed in claim 8, wherein the first and second signal portions are temporally interleaved with each other.

10. The ultrasound therapy apparatus as claimed in claim 9, wherein the oscillatory wave signal is a continuous signal.

11. The ultrasound therapy apparatus as claimed in claim 9, wherein the oscillatory wave signal is a discontinuous signal.

12. The ultrasound therapy apparatus as claimed in claim 1, wherein the oscillatory wave signal has a fixed amplitude.

13. The ultrasound therapy apparatus as claimed in claim 1, wherein the frequency of the ultrasound energy corresponds to that of the driving signal.
